(19) United States
(12) Patent Application Publication
(10) Pub. No.: US 2003/0037344 A1
(43) Pub. Date: Feb. 20, 2003

(54) METHOD FOR EVALUATING THE ABILITY OF A COMPOUND TO INHIBIT NEUROTOXICITY

(76) Inventors: DAVID STERN, GREAT NECK, NY (US); SHI DU YAN, NEW YORK, NY (US); BENJAMIN WOLOZIN, HINSDALE, IL (US)

Correspondence Address: JOHN P WHITE, COOPER & DURHAM, 1185 AVENUE OF THE AMERICAS, NEW YORK, NY, 10036

(21) Appl. No.: 08/997,464

(22) Filed: Dec. 23, 1997

(51) Int. Cl. A61K 49/00; A01K 67/00

(52) U.S. Cl. 800/3; 800/8

(57) ABSTRACT

This invention provides a method for evaluating the ability of a compound to inhibit neurotoxicity which comprises (a) contacting a cell which expresses a receptor for advanced glycation end product protein and a mutant presenilin-2 protein in a cell culture and the compound; (b) determining the level of cell death in the cell culture; and (c) comparing the level of cell death determined in step (b) with the amount determined in the absence of the compound so as to evaluate the ability of the compound to inhibit neurotoxicity.

The invention also provides a method for evaluating the ability of a compound to inhibit binding of an amyloid-β peptide to a receptor for advanced glycation end product which comprises (a) contacting a cell which expresses a mutant presenilin-2 protein and a receptor for advanced glycation end product protein with amyloid-β protein and the compound; (b) determining the amount of amyloid-β peptide bound to the cell; (c) comparing the amount of bound amyloid-β peptide determined in step (b) with the amount determined in the absence of the compound so as to evaluate the ability of the compound to inhibit binding of the amyloid-β peptide to the receptor for advanced glycation end product.

The invention also provides a transgenic non-human animal whose somatic and germ cells express mutant human presenilin-2 protein and human receptor for advanced glycation end product protein.

The invention disclosed herein was made with Government support from National Institute of Aging/National Institutes of Health grants K01AG00690, RO1AG14103, and R01HL56881. Accordingly, the U.S. Government has certain rights to this invention.

### BACKGROUND OF THE INVENTION

Throughout this application, various publications are referenced by author and date. Full citations for these publications may be found listed alphabetically at the end of the specification immediately preceding the claims. The disclosures of these publications in their entireties are hereby incorporated by reference in order to more fully describe the state of the art.

Neurologic disease represents a particularly problematic area for the identification of therapeutic compounds which are effective in humans. Alzheimer's disease (AD), for example, has come under intense scrutiny, but no effective therapies have yet been identified. There is a critical need for an effective system by which therapeutic compounds can be identified.

Extracellular accumulations of amyloid in neuritic plaques composed predominately of amyloid-beta peptide (Aβ) are pathognomonic features of AD (Haas et al., 1994; Kosik, et al., 1994; Yankner, et al., 1996; Goedert, et al., 1993; Trojanowski, et al., 1994). These lesions increase in number and volume over time resulting in an apparent replacement of the neuronal cell population (Haas et al., 1994; Kosik, et al., 1994; Yankner, et al., 1996; Goedert, et al., 1993; Trojanowski, et al., 1994; Cummings, et al., 1995.), and are closely associated with neuronal toxicity leading to dementia.

In AD it is widely accepted that later in the course of the disease, when Aβ fibrils are abundant, nonspecific interactions of such fibrils with the cell surface may be frequent and disruptive for cellular functions(Yankner, B., et al., 1990; Cotman, et al. 1995; Mattson, et al., 1995; Hensley, et al., 1994; Behl, et al., 1994; Younkin, et al., 1995). Aβ fibrils can disrupt plasma membranes, causing changes in course of the disease, when Aβ fibrils are present at lower levels (and monomers/oligomers predominate, as opposed to fibrils), higher affinity interactions with cellular surfaces are more likely to be relevant. The immunoglobulin superfamily receptor RAGE (Receptor for Advanced Glycation Endproduct), expressed by neurons and microglia, is present at high levels in AD brain, both in areas of affected brain parenchyma (at the antigen and mRNA levels) and in nearby vasculature. RAGE is a receptor with nanomolar affinity for Aβ monomer/oligomer, as well as for fibrils (Yan, et al., 1996). In culture, cells expressing RAGE display enhanced susceptibility to Aβ-induced cellular dysfunction compared with those expressing lower levels of RAGE, or those in which the receptor is blocked. Consistent with a role for Aβ-receptor interactions in early perturbation of neuronal functions, relevant outcomes of Aβ binding to neuronal RAGE include activation of nuclear factor-KB (NF-kB), induction of heme oxygenase type 1 and expression of macrophage-colony stimulating factor (M-CSF), each of which can be demonstrated in AD brain (Yan, et al., 1996; Yan, et al. 1997).

Mutant presenilins 1 and 2 are closely associated with most cases of early onset familial AD (Haas, et al., 1996; Dwji, et al., 1996; Tanzi, et al., 1996; Hardy, et al. 1997). Furthermore, presenilin-2 may be involved in cellular pathways which eventuate in programmed cell death; a mutant form of presenilin-2 results in expression of a molecule causing increased basal apoptosis in nerve growth factor-differentiated PC12 cells (Wolozin, et al., 1996). PC12 cells, stably transfected with amyloid precursor protein (APP) show increased levels of apoptosis after serum withdrawal. Cellular stress increases synthesis of APP and, depending on the particular stress, secretion of either APP of Aβ. Enhanced activation of presenilin-2 protein expression might not only increase the tendency toward apoptosis, but by activating apoptotic signals, may trigger a stress response and increase production of Aβ, leading to neurodegeneration (Wolozin, et al., 1996).

### SUMMARY OF THE INVENTION

The present invention provides a method for evaluating the ability of a compound to inhibit neurotoxicity which comprises (a) contacting a cell which expresses a receptor for advanced glycation end product protein and a mutant presenilin-2 protein in a cell culture with the compound; (b) determining the level of cell death in the cell culture; and (c) comparing the level of cell death determined in step (b) with the amount determined in the absence of the compound so as to evaluate the ability of the compound to inhibit neurotoxicity.

Additionally, the present invention provides a method for evaluating the ability of a compound to inhibit binding of an amyloid-β peptide to a receptor for advanced glycation end product which comprises (a) contacting a cell which expresses a mutant presenilin-2 protein and a receptor for advanced glycation end product protein with amyloid-β protein and the compound; (b) determining the amount of amyloid-β peptide bound to the cell; (c) comparing the amount of bound amyloid-β peptide determined in step (b) with the amount determined in the absence of the compound so as to evaluate the ability of the compound to inhibit binding of the amyloid-β peptide to the receptor for advanced glycation end product.

The present invention also provides a pharmaceutical composition which comprises a compound capable of inhibiting neurotoxicity and a pharmaceutically acceptable carrier.

Moreover, the present invention additionally provides a method for treating a neurodegenerative condition in a subject which comprises administering to the subject an amount of a pharmaceutical composition, effective to treat the neurodegenerative condition in the subject.

Further, the present invention also provides a transgenic non-human animal whose somatic and germ cells contain and express a gene encoding mutant human presenilin-2 protein and whose somatic and germ cells contain and express a gene encoding human receptor for advanced glycation end product protein, the genes having been introduced into the animal or an ancestor of the animal at an embryonic stage and wherein the gene may be operably linked to an inducible promoter element.

The invention further provides a method for identifying whether a compound is capable of ameliorating a neurodegenerative condition in an animal comprising (a) administering the compound to a transgenic animal, wherein the animal exhibits a neurodegenerative condition; (b) measuring the level of neurodegeneration in the animal; and (c) comparing the level of neurodegeneration measured in step (b) with the level of neurodegeneration measured in the animal in the absence of the compound so as to identify whether the compound is capable of ameliorating the neurodegenerative condition in the animal.

Finally, the invention provides a cell comprising a recombinant nucleic acid encoding mutant presenilin-2 protein and encoding receptor for advanced glycation endproduct protein.

### BRIEF DESCRIPTION OF THE FIGURES

FIGS. 1A-1B. Immunostaining of stably RAGE-transfected (A) or mock-transfected (B) PC12 cells. These experiments employed anti-human RAGE IgG and methods described in Yan et al., 1996.

FIG. 2. Immunoblotting was performed on extracts of stably RAGE-transfected (lane 2) or mock-transfected (lane 1) PC12 cells. These experiments employed anti-human RAGE IgG and methods described in Yan et al., 1996.

FIG. 3. Stably RAGE-transfected PC12 cells or mock-transfected controls were co-transfected with presenilin 2 (RAGE/PS2) and exposed to the indicated concentration of Aβ, as indicated. Apoptosis was determined 18 hours. later by TUNEL staining.

### DETAILED DESCRIPTION OF THE INVENTION

This invention provides a method for evaluating the ability of a compound to inhibit neurotoxicity which comprises (a) contacting a cell which expresses a receptor for advanced glycation end product protein and a mutant presenilin-2 protein in a cell culture with the compound; (b) determining the level of cell death in the cell culture; and (c) comparing the level of cell death determined in step (b) with the amount determined in the absence of the compound so as to evaluate the ability of the compound to inhibit neurotoxicity.

This invention also provides a method for evaluating the ability of a compound to inhibit cytotoxicity in a non-neuronal cell which comprises (a) contacting a cell which expresses a receptor for advanced glycation end product protein and a mutant presenilin-2 protein in a cell culture with the compound (b) determining the level of cell death in the cell culture; and (c) comparing the level of cell death determined in step (b) with the amount determined in the absence of the compound so as to evaluate the ability of the compound to inhibit cytotoxicity.

In this embodiment, the cell may be contacted with the compound and amyloid-β peptide. The cell may be contacted with amyloid-β peptide simultaneously or the cell may be contacted with amyloid-β peptide and the compound at different times. The compound may be capable of specifically binding to amyloid-β peptide. The compound may bind to amyloid-β peptide at the site where the receptor for advanced glycation end product interacts. The compound may be a soluble extracellular portion of a receptor for advanced glycation end product. The compound may be bound to a solid support. The compound may be expressed on the surface of a cell. The compound may be present on the surface of a cell.

In the present invention the cell may be a neuronal cell, an endothelial cell, a glial cell, a microglial cell, an astrocyte, a smooth muscle cell, a somatic cell, a bone marrow cell, a liver cell, an intestinal cell, a germ cell, a myocyte, a mononuclear cell, a mononuclear phagocyte, a tumor cell, a stem cell, or a PC12 cell. The cell may be under oxidant stress. The compound may be a peptide, a peptidomimetic, a nucleic acid, a polymer, or a small molecule. The compound may be bound to a solid support. The presenilin-2 may be a mutant or non-mutant form of presenilin-2. The mutant form of presenilin-2 may be in the form of a deletion, substitution, insertion, or point mutation. The presenilin-2 may be of human or non-human origin. The mutant presenilin-2 protein may be overexpressed. The receptor for advanced glycation end product may be overexpressed.

One embodiment of this invention is a method for evaluating the ability of a compound to inhibit binding of an amyloid-β peptide to a receptor for advanced glycation end product which comprises (a) contacting a cell which expresses a mutant presenilin-2 protein and a receptor for advanced glycation end product protein with amyloid-β, protein and the compound; (b) determining the amount of amyloid-β peptide bound to the cell; (c) comparing the amount of bound amyloid-β peptide determined in step (b) with the amount determined in the absence of the compound so as to evaluate the ability of the compound to inhibit binding of the amyloid-β peptide to the receptor for advanced glycation end product.

In this embodiment, the cell may be contacted with the compound and the amyloid-β peptide simultaneously or the cell may be contacted with the amyloid-β peptide and the compound at different times. The compound may be capable of specifically binding to amyloid-β peptide. The compound may bind to amyloid-β peptide at the site where the receptor for advanced glycation end product interacts. The compound may be a soluble extracellular portion of a receptor for advanced glycation end product. The compound may be bound to a solid support. The compound may be expressed on the surface of a cell. The compound may be present on the surface of a cell.

In this embodiment, the cell may be a neuronal cell, an endothelial cell, a glial cell, a microglial cell, an astrocyte, a smooth muscle cell, a somatic cell, a bone marrow cell, a liver cell, an intestinal cell, a germ cell, a myocyte, a mononuclear cell, a mononuclear phagocyte, a tumor cell, a stem cell, or a PC12 cell. The compound may be a peptide, a peptidomimetic, a nucleic acid, a polymer, or a small molecule. The compound may be bound to a solid support. The presenilin-2 may be a mutant or non-mutant form of presenilin-2. The mutant form of presenilin-2 may be in the form of a deletion, substitution, insertion, or point mutation. The presenilin-2 may be of human or non-human origin. The mutant presenilin-2 protein may be overexpressed. The receptor for advanced glycation end product may be overexpressed.

Another embodiment of the present invention provides for a pharmaceutical composition which comprises a compound capable of inhibiting neurotoxicity, and a pharmaceutically acceptable carrier. The carrier may be a diluent, an aerosol, a topical carrier, an aquous solution, a nonaqueous solution or a solid carrier.

Another embodiment of this invention provides for a method for treating a neurodegenerative condition in a subject which comprises administering to the subject an amount of the provided pharmaceutical composition, effective to treat the neurodegenerative condition in the subject. The subject may be a mammal or a human. The administration in this embodiment may be intralesional, intraperitoneal, intramuscular, or intravenous injection; infusion; liposome mediated delivery; topical, nasal, oral, anal, ocular or otic delivery.

In this embodiment, the neurodegenerative condition may be associated with Alzheimer's disease, diabetes, senility, renal failure, hyperlipidemic atherosclerosis, neuronal cytoxicity, Down's syndrome, dementia associated with head trauma, amyotrophic lateral sclerosis, myasthenia gravis, multiple sclerosis or neuronal degeneration. The neurodegenerative condition may be associated with spongiform encephalopathic disease, including but not limited to Creutzfeldt-Jakob Disease, Fatal Familial Insomnia, kuru, Gerstmann-Straussler-Scheinker Disease, bovine spongiform encephalopathy, feline spongiform encephalopathy, transmissible mink encephalopathy, zoological spongiform encephalopathy, Alper's Disease or scrapie. The neurodegenerative condition may be associated with degeneration of a neuronal cell in the subject. The neurodegenerative condition may be associated with the formation of an amyloid-β peptide fibril. The neurodegenerative condition may be associated with aggregation of amyloid-β peptide. The neurodegenerative condition may be due to oxidant or cellular stress. The neurodegenerative condition may be associated with infiltration of a microglial cell into a senile plaque. The neurodegenerative condition may be associated with activation of a microglial cell by an amyloid-β peptide.

Still another embodiment of the present invention provides for a transgenic non-human animal whose somatic and germ cells contain and express a gene encoding mutant human presenilin-2 protein and whose somatic and germ cells contain and express a gene encoding human receptor for advanced glycation end product protein, the genes having been introduced into the animal or an ancestor of the animal at an embryonic stage and wherein the gene may be operably linked to an inducible promoter element. The transgenic animal may be a mouse or other non-human mammal. The mutant presenilin-2 protein may be overexpressed. The receptor for advanced glycation end product may be overexpressed. The presenilin-2 may be a mutant or non-mutant form of presenilin-2. The mutant form of presenilin-2 may be in the form of a deletion, substitution, insertion, or point mutation. The presenilin-2 may be of human or non-human origin.

Yet another embodiment of the present invention provides for a screening method to identify drugs, compounds or agents capable of treating symptoms associated with neurotoxicity wherein the mechanism of cell death is modeled by the cell culture or the transgenic animal. The screening method may be performed on a library of drugs, compounds or agents. The screening method may involved mass screening, large throughput, automated or robotic processing and analysis, or combinations thereof.

In one embodiment of this invention, the screening method is provided as part of a screening kit. A screening kit may include a cell which expresses a receptor for advanced glycation end product protein and a mutant or non-mutant presenilin protein. Additionally, a screening kit may include a cell which does not express receptor for advanced glycation end product protein or a presenilin protein. The kit may include a cell which expresses receptor for advanced glycation end product protein but not presenilin protein or a cell which expresses a presenilin protein but not a receptor for advanced glycation end product protein. A kit may also include buffers and reagents for the detection and measurement of cell death, cell lysis, cell viability, apoptosis, or other cellular functions. Additionally a kit may include a solid support matrix.

An additional embodiment of the present invention provides a cell isolated from the transgenic animal which expresses a transgene encoding mutant presenilin-2 protein and a transgene encoding a receptor for advanced glycation end product protein. The isolated cell may be a neuronal cell, a glial cell, a microglial cell, an astrocyte, an endothelial cell, a mononuclear cell, a tumor cell, a muscle cell, a bone marrow cell, a liver cell, an intestinal cell, a germ cell, a myocyte, a mononuclear phagocyte, or a stem cell.

Another embodiment of the present invention provides for a method for identifying whether a compound is capable of ameliorating a neurodegenerative condition in an animal comprising (a) contacting a cell isolated from a transgenic animal which expresses a transgene encoding mutant presenilin-2 protein and a transgene encoding a receptor for advanced glycation end product protein with amyloid-β protein and the compound; (b) determining the amount of amyloid-β peptide bound to the cell; (c) comparing the amount of bound amyloid-β peptide determined in step (b) with the amount determined in the absence of the compound so as to evaluate the ability of the compound to inhibit binding of the amyloid-β peptide to the receptor for advanced glycation end product.

Another embodiment of the present invention provides for a method for identifying whether a compound is capable of ameliorating a neurodegenerative condition in an animal comprising (a) contacting a cell isolated from a transgenic animal which expresses a gene encoding mutant presenilin-2 protein and which expresses a gene encoding a receptor for advanced glycation end product protein with the compound; (b) determining the level of cell death; (c) comparing the level of cell death determined in step (b) with the level determined in the absence of the compound so as to evaluate the ability of the compound to inhibit neurotoxicity.

Another embodiment of the present invention provides for a method for identifying whether a compound is capable of ameliorating a neurodegenerative condition in an animal comprising (a) administering the compound to the provided transgenic animal, wherein the animal exhibits a neurodegenerative condition; (b) measuring the level of neurodegeneration in the animal; and (c) comparing the level of neurodegeneration measured in step (b) with the level of neurodegeneration measured in the animal in the absence of the compound so as to identify whether the compound is capable of ameliorating the neurodegenerative condition in the animal. The administration in this embodiment may be intralesional, intraperitoneal, intramuscular, or intravenous injection; infusion; liposome mediated delivery; topical, nasal, oral, anal, ocular or otic delivery.

In this embodiment, the neurodegenerative condition may be associated with Alzheimer's disease, diabetes, senility, renal failure, hyperlipidemic atherosclerosis, neuronal cytoxicity, Down's syndrome, dementia associated with head trauma, amyotrophic lateral sclerosis, myasthenia gravis, multiple sclerosis or neuronal degeneration. The neurodegenerative condition may be associated with spongiform encephalopathic disease, including but not limited to Creutzfeldt-Jakob Disease, Fatal Familial Insomnia, kuru, Gerstmann-Straussler-Scheinker Disease, bovine spongiform encephalopathy, feline spongiform encephalopathy, transmissible mink encephalopathy, zoological spongiform encephalopathy, Alper's Disease or scrapie. The neurodegenerative condition may be associated with degeneration of a neuronal cell in the subject. The neurodegenerative condition may be associated with the formation of an amyloid-β peptide fibril. The neurodegenerative condition may be associated with aggregation of amyloid-β peptide. The neurodegeneration may be due to oxidant or cellular stress. The neurodegenerative condition may be associated with infiltration of a microglial cell into a senile plaque. The neurodegenerative condition may be associated with activation of a microglial cell by an amyloid-β peptide.

As used herein, the term “oxidant stress” encompasses the perturbation of the ability of a cell to ameliorate the toxic effects of oxidants. Oxidants may include hydrogen peroxide or oxygen radicals that are capable of reacting with bases in the cell including DNA. A cell under oxidant stress may undergo biochemical, metabolic, physiological and/or chemical modifications to counter the introduction of such oxidants. Such modifications may include lipid peroxidation, NF-kB activation, heme oxygenase type I induction and DNA mutagenesis. Also, antioxidants such as glutathione are capable of lowering the effects of oxidants. “Cellular stress” may also be induced by serum starvation or by the withdrawal or deprivation of other trophic factors which may perturb normal cellular function. Such perturbations may be by the same or by different mechanisms as that induced by oxidant stress.

As used herein, apoptotic cell death is programmed or gene-directed cell death. A hallmark of apoptosis is the activation of endonuclease that attacks cellular genomic DNA at the linker regions that connect nucleosomal units. Degradation of DNA ensues, producing DNA fragments that can be observed as a distinct DNA ladder pattern.

As used herein, the term “neurotoxicity” encompasses the negative metabolic, biochemical and physiological effects on a neuronal cell which may result in a debilitation of the neuronal celluar functions, including but not limited to neuronal cell death. Such functions may include memory, learning, perception, neuronal electrophysiology (ie. action potentials, polarizations and synapses), synapse formation, both chemical and electrical, channel functions, neurotransmitter release and detection and neuromotor functions. Neurotoxicity may include neuronal cytotoxicity or neuronal cell death.

As used herein, the term “neuronal degeneration” encompasses a decline in normal functioning of a neuronal cell. Such a decline may include a decline in memory, learning, perception, neuronal electrophysiology (ie. action potentials, polarizations and synapses), synapse formation, both chemical and electrical, channel functions, neurotransmitter release and detection and neuromotor functions. In the present invention, the subject may be a mammal or a human subject.

As used herein, the term “cytotoxicity” encompasses the negative metabolic, biochemical and physiological effects on a cell which may result in a debilitation of the celluar functions, including but not limited to cell death.

In the practice of any of the methods of the invention or preparation of any of the pharmaceutical compositions an “therapeutically effective amount” is an amount which is capable of inhibiting the binding of an amyloid-β peptide with a receptor for advanced glycation eudproduct. Accordingly, the effective amount will vary with the subject being treated, as well as the condition to be treated. For the purposes of this invention, the methods of administration are to include, but are not limited to, administration cutaneously, subcutaneously, intravenously, parenterally, orally, topically, or by aerosol.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

This invention also provides for pharmaceutical compositions capable of inhibiting neurotoxicity together with suitable diluents, preservatives, solubilizers, emulsifiers, adjuvants and/or carriers. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition.

Controlled or sustained release compositions include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

When administered, compounds are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Attachment of polyethylene glycol (PEG) to compounds is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous compounds. For example, a PEG adduct of a human protein might be useful for the treatment of disease in other mammalian species without the risk of triggering a severe immune response. The carrier includes a microencapsulation device so as to reduce or prevent an host immune response against the compound or against cells which may produce the compound. The compound of the present invention may also be delivered microencapsulated in a membrane, such as a liposome.

Polymers such as PEG may be conveniently attached to one or more reactive amino acid residues in a protein such as the alpha-amino group of the amino terminal amino acid, the epsilon amino groups of lysine side chains, the sulfhydryl groups of cysteine side chains, the carboxyl groups of aspartyl and glutamyl side chains, the alphacarboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

The pathologic hallmarks of Alzheimer's disease (AD) are intracellular and extracellular deposition of filamentous proteins which closely correlates with eventual neuronal dysfunction and clinical dementia (for reviews see Goedert, 1993; Haas et al., 1994; Kosik, 1994; Trojanowski et al., 1994; Wischik, 1989). Amyloid-β peptide (Aβ) is the principal component of extracellular deposits in AD, both in senile/diffuse plaques and in cerebral vasculature. Aβ has been shown to promote neurite outgrowth, generate reactive oxygen intermediates (ROIs), induce cellular oxidant stress, lead to neuronal cytotoxicity, and promote microglial activation (Behl et al., 1994; Davis et al., 1992; Hensley, et al., 1994; Koh, et al., 1990; Koo et al., 1993; Loo et al., 1993; Meda et al., 1995; Pike et al., 1993; Yankner et al., 1990). For Aβ to induce these multiple cellular effects, it is likely that plasma membranes present a binding protein(s) which engages Aβ.

A link between cell death, mutant presenilins, and RAGE is proposed. An investigation into whether increased expression of RAGE promotes cellular interactions with Aβ, thereby increasing cell stress and, in the presence of mutant presenilin 2, synergistically drives cells into apoptosis was performed. As mutant presenilin and elevated levels of RAGE are both associated with AD, the interaction of these two molecules, either directly or indirectly, might greatly augment Aβ toxicity.

This invention is illustrated by examples set forth in the Experimental Details section which follows. This section is provided to aid in an understanding of the invention but is not intended to, and should not be construed to, limit in any way the invention as set forth in the claims which follow thereafter.

Experimental Details

### EXAMPLE 1

### Presenilin-2 Enhances Cytotoxicity Due to Amyloid-β Peptide Interaction With RAGE on Neurons

Introduction

Extracellular accumulations of amyloid in neuritic plaques composed predominately of amyloid-beta peptide (Aβ) are pathognomonic features of Alzheimer's disease (AD) (Haas et al., 1994; Kosik, et al., 1994; Yankner, et al., 1996; Goedert, et al., 1993; Trojanowski, et al., 1994). These lesions increase in number and volume over time resulting in an apparent replacement of the neuronal cell population(Haas et al., 1994; Kosik, et al., 1994; Yankner, et al., 1996; Goedert, et al., 1993; Trojanowski, et al., 1994; Cummings, et al., 1995), and are closely associated with neuronal toxicity leading to dementia.

In AD it is widely accepted that later in the course of the disease, when Aβ fibrils are abundant, nonspecific interactions of such fibrils with the cell surface may be frequent and disruptive for cellular functions(Yankner, et al., 1990; Cotman, et al. 1995; Mattson, et al., 1995; Hensley, et al., 1994; Behl, et al., 1994; Younkin, et al., 1995). Aβ fibrils can disrupt plasma membranes, causing changes in course of the disease, when Aβ fibrils are present at lower levels (and monomers/oligomers predominate, as opposed to fibrils), higher affinity interactions with cellular surfaces are more likely to be relevant. The immunoglobulin superfamily receptor RAGE (receptor for advanced glycation end product), expressed by neurons and microglia, is present at high levels in AD brain, both in areas of affected brain parenchyma (at the antigen and mRNA levels) and in nearby vasculature. RAGE is a receptor with nanomolar affinity for Aβ monomer/oligomer, as well as for fibrils (Yan, et al., 1996). In culture, cells expressing RAGE display enhanced susceptibility to Aβ-induced cellular dysfunction compared with those expressing lower levels of RAGE, or those in which the receptor is blocked. Consistent with a role for Aβ-receptor interactions in early perturbation of neuronal functions, relevant outcomes of Aβ binding to neuronal RAGE include activation of nuclear factor-KB (NF-kB), induction of heme oxygenase type 1 and expression of macrophage-colony stimulating factor (M-CSF), each of which can be demonstrated in AD brain (Yan, et al., 1996; Yan, et al. 1997).

Mutant presenilins 1 and 2 are closely associated with early onset familial AD (Haas, et al., 1996; Dwji, et al., 1996; Tanzi, et al., 1996; Hardy, et al. 1997). Furthermore, a relationship between presenilin-2 and cellular pathways eventuating in programmed cell death is indicated; a mutant form of presenilin-2 results in expression of a molecule causing increased basal apoptosis in nerve growth factor-differentiated PC12 cells (Wolozin et al., 1996).

Results

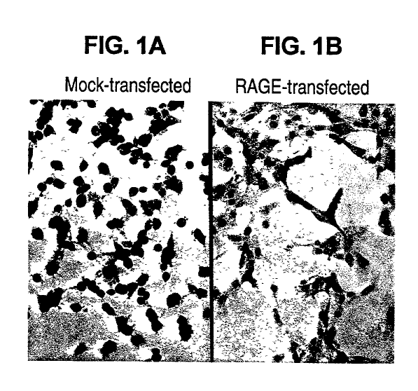

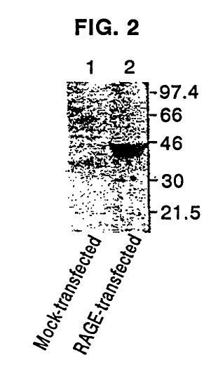

Characterization of Transfected Cells. PC12 cells stably transfected to overexpress RAGE showed increased levels of RAGE, compared with nontransfected controls, by immunostaining (FIGS. 1A-1B). Furthermore, immunoblotting of cell extracts demonstrated increased RAGE antigen in RAGE tranfectants versus mock-transfected controls (FIG. 2). Transfection of cells with the mutant presenilin 2 construct has been shown to result in overexpression of presenilin-2 antigen (Wolozin et al., 1996).

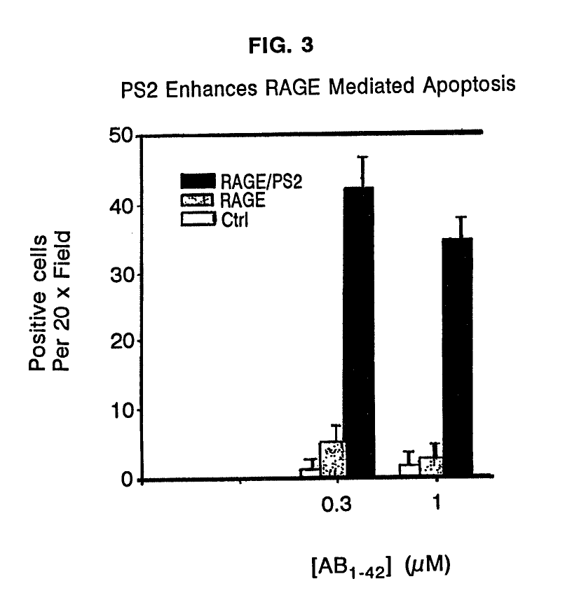

Induction of apoptosis. For apoptosis studies, PC12 cultures, either mock-transfected (control), stably transfected with the RAGE-bearing construct or stably transfected with the RAGE-bearing construct and transiently transfected with the mutant presenilin-2 bearing construct, and were then exposed to Aβ (0.3 or 1 μM) for 24 hrs. At the end of this time, apoptosis was determined using the TUNEL assay by counting positively staining nuclei per twenty high-power fields. Under these conditions, mutant presenilin-2 by itself has little effect on apoptosis, as shown previously (FIG. 3). However, cells co-transfected to express mutant presenilin-2 and RAGE showed a dramatic increase in apoptosis at Aβ concentrations of both 0.3 and 1 μM.

Discussion

Alzheimer's disease is likely to result from a combination of factors resulting in increased production of Aβ, enhanced susceptibility of cells to the effects of Aβ, and an augmented apoptotic response to environmental stimuli. RAGE tethers Aβ to the cell surface; this led us to consider the hypothesis that increased RAGE expression, along with elevated levels of mutant presenilin-2, might enhance Aβ toxicity. As demonstrated herein, this general concept proved to be true. However, the synergistic interaction of these two factors resulted in dramatically increased apoptosis; the latter suggesting a potent mechanism for inducing neuronal death in Alzheimer's disease. The interaction of mutant presenilin-2 with RAGE in transfected cultured cells, as well as in transgenic mice, provides a useful model system for investigating the pathobiology of AD and as a model system for identifying and testing neuroprotective therapeutics.

Experimental Procedures

Generation of Stable Transfected Cells. PC12 cells, grown as described and transfected with pcDNA3-RAGE (the latter comprised of the human RAGE cDNA) (Yan, et al., 1996) using lipofectamine (12 μg/ml) according to the manufacturer's instructions (Gibco-BRL). Cells were maintained in the presence of G418. Transient transfection was performed with a vector including mutant presenilin-2 (N141 mutant) (Wolozin et al., 1996), using lipofectamine, as described (Wolozin et al., 1996). Immunoblotting and immunostaining of PC12 cells for RAGE was performed as described previously (Yan, et al., 1996).

Apoptosis assay. The terminal deoxynucleotidyl transferase-mediated dUTP nick end labeling (TUNEL) assay was performed as described using a kit from Travigen, a peroxide-based TACS-TdTkit (Woolozin, et al., 1996). Aβ (comprised of residues 1-42) was synthesized, HPLC purified, and purchased from a commercial supplier.

### REFERENCES

Abuchowski, A, et al., Immunosuppressive properties and circulating life of Achromobacter glutaminase-asparaginase covalently attached to polyethylene glycol in man. Cancer Treat Rep. 65:1077-81 (1981).

Behl, C., et al. Hydrogen peroxide mediates amyloid Aβ protein toxicity. Cell 77. 817-827(1994).

Carpenter, C. P., et al. Response of dogs to repeated intravenous injection of polyethylene glycol 4000 with notes on excretion and sensitization. Toxicol. Appl. Pharmacol. 18:35-40 (1971).

Cotman, C. and Anderson, A. A potential role for apoptosis in neurodegeneration and Alzheimer's disease. Mol. Neurobiol. 10:19-45 (1995).

Cummings, B. and Cotman, C. Image analysis of β-amyloid load in Alzheimer's disease and relation to dementia severity. Lancet 346:1524-1428 (1995).

Davis, J., et al., BBRC 189:1096-1100 (1992).

Dwji, N. and Singer, S. Genetic clues to Alzheimer's disease. Science 271:159-160 (1996).

Goedert, M. Tau protein and the neurofibrillary pathology of Alzheimer's disease. Trends Neurosci. 16:460-465 (1993).

Haas, C. and Selkoe, D. Cellular processing of β-amyloid precursor protein and the genesis of amyloid β-peptide. Cell 75: 1039-1042 (1994).

Haas, C. Presenile because of presenilin: the presenilin genes and early onset Alzheimer's disease. Curr. Opin. Neurol. 9: 254-259 (1996).

Hardy, J. Amyloid, the presenilins and Alzheimer's disease. Trends Neurosci. 20: 154-159 (1997).

Hensley, K., et al. A model for β-amyloid aggregation and neurotoxicity based on free radical generation by the peptide; relevance to Alzheimer's disease. Proc. Natl. Acad. Sci. USA 91: 3270-3274 (1994).

Katre, N. V., et al. Chemical modification of recombinant interleukin 2 by polyethylene glycol increases its potency in the murine Meth A sarcoma model. Proc. Natl. Acad. Sci. USA 84:1487-91 (1987).

Koh, J. Y., et al. Beta-amyloid protein increases the vulnerability of cultured cortical neurons to excitotoxic damage. Brain Res. 533:315-320 (1990)

Koo, E. H., et al. Amyloid beta-protein as a substrate interacts with extracellular matrix to promote neurite outgrowth. PNAS(USA) 90:4748-4752 (1993).

Kosik, K. S. Alzheimer's disease sphinx: a riddle with plaques and tangles. J. Cell Biol. 127: 1501-1504 (1994).

Loo, D. T., et al. Apoptosis is induced by beta-amyloid in cultured central nervous system neurons. PNAS(USA) 90:7951-7955 (1993).

Mattson, M. P. Free radicals and disruption of neuronal ion homeostasis in AD: a role for amyloid beta-peptide? Neurobiol. Aging 16 661-674 (1995).

Meda, L., et al. Activation of microglial cells by beta-amyloid protein and interferon-gamma. Nature 374:647-650 (1995).

Newmark et al. 1982.

Pike, C. J., et al. Neurodegeneration induced by beta-amyloid peptides in vitro: the role of peptide assembly state. J Neurosci. 13:1676-1687 (1993).

Tanzi, R., et al. The gene defects responsible for familial Alzheimer's disease. Neurobiol. Dis. 3: 159-168 (1996).

Trojanowski, J. and Lee, V. Paired helical filament tau in Alzheimer's Disease, the knase connection. Am. J. Pathol. 1441: 449-453 (1994).

Wischik, C. Cell biology of the Alzheimer tangle. Curr. Opin. Cell Biol. 1, 115-122 (1989).

Wolozin, B., et al. Participation of PS2 in apoptosis: enhanced basal activity conferred by an Alzheimer mutation. Science 274: 1710-1713 (1996).

Yan S. D., et al. Amyloid-β peptide RAGE interaction elicits neuronal expression of M-CSF: a proinflammatory pathway in Alzheimer's disease. Proc. Natl. Acad. Sci. USA 94: 5296-5301 (1997).

Yan, S. D., et al. RAGE and amyloid-Aβ peptide neurotoxicity in Alzheimer's disease. Nature 382: 685-691 (1996).

Yankner, B., et al. Neurotrophic and neurotoxic effects of amyloid β protein: reversal by tachykinin neuropeptides. Science 250: 279-282 (1990).

Yankner, B. Mechanisms of neuronal degeneration as in Alzheimer's disease. Neuron 16: 921-932 (1996).

Younkin, S. Evidence that Aβ is the real culprit in Alzheimer's disease. Ann. Neurol. 37: 287-288 (1995).

What is claimed is:

1. A method for evaluating the ability of a compound to inhibit neurotoxicity which comprises:
(a) contacting a cell which expresses a receptor for advanced glycation end product protein and a mutant presenilin-2 protein in a cell culture and the compound;
(b) determining the level of cell death in the cell culture; and
(c) comparing the level of cell death determined in step (b) with the amount determined in the absence of the compound so as to evaluate the ability of the compound to inhibit neurotoxicity.

2. The method of claim 1, wherein the cell is a neuronal cell, a glial cell, a microglial cell, an astrocyte, an endothelial cell, a mononuclear cell, a tumor cell, or a PC12 cell.

3. The method of claim 1, wherein the compound is a peptide, a peptidomimetic, a nucleic acid, a polymer, or a small molecule.

4. The method of claim 1, wherein the compound is bound to a solid support.

5. The method of claim 1, wherein the mutant presenilin-2 is overexpressed.

6. A method for evaluating the ability of a compound to inhibit binding of an amyloid-β peptide to a receptor for advanced glycation end product which comprises:
(a) contacting a cell which expresses a mutant presenilin-2 protein and a receptor for advanced glycation end product protein with amyloid-β protein and the compound;
(b) determining the amount of amyloid-β peptide bound to the cell;
(c) comparing the amount of bound amyloid-β peptide determined in step (b) with the amount determined in the absence of the compound so as to evaluate the ability of the compound to inhibit binding of the amyloid-β peptide to the receptor for advanced glycation end product.

7. The method of claim 1, wherein the cell is a neuronal cell, a glial cell, a microglial cell, an astrocyte, an endothelial cell, a mononuclear cell, a tumor cell, or a PC12 cell.

8. The method of claim 1, wherein the compound is a peptide, a peptidomimetic, a nucleic acid, a polymer, or a small molecule.

9. The method of claim 1, wherein the compound is bound to a solid support.

10. The method of claim 1, wherein the mutant presenilin-2 is overexpressed.

11. A pharmaceutical composition which comprises a compound capable of inhibiting neurotoxicity identified by the method of claim 1, and a pharmaceutically acceptable carrier.

12. The pharmaceutical composition of claim 11, wherein the carrier is a diluent, an aerosol, a topical carrier, an aqueous solution, a nonaqueous solution or a solid carrier.

13. A method for treating a neurodegenerative condition in a subject which comprises administering to the subject an amount of the pharmaceutical composition of claim 11, effective to treat the neurodegenerative condition in the subject.

14. The method of claim 13, wherein the neurodegenerative condition is associated with Alzheimer's disease, diabetes, senility, renal failure, hyperlipidemic atherosclerosis, neuronal cytoxicity, Down's syndrome, dementia associated with head trauma, amyotrophic lateral sclerosis, myasthenia gravis, multiple sclerosis or neuronal degeneration.

15. The method of claim 13, wherein the neurodegenerative condition is associated with degeneration of a neuronal cell in the subject.

16. The method of claim 13, wherein the neurodegenerative condition is associated with the formation of an amyloid-β peptide fibril.

17. The method of claim 13, wherein the neurodegenerative condition is associated with aggregation of amyloid-β peptide.

18. The method of claim 13, wherein the neurodegenerative condition is associated with infiltration of a microglial cell into a senile plaque.

19. The method of claim 13, wherein the neurodegenerative condition is associated with activation of a microglial cell by an amyloid-β peptide.

20. The method of claim 13, wherein the subject is a human.

21. A transgenic non-human animal whose somatic and germ cells contain and overexpress a gene encoding human presenilin-2 protein and whose somatic and germ cells contain and overexpress a gene encoding human receptor for advanced glycation end product protein, the genes having been introduced into the animal or an ancestor of the animal at an embryonic stage and wherein the gene may be operably linked to an inducible promoter element.

22. The animal of claim 21, wherein the animal is a mouse.

23. The animal of claim 21, wherein the gene encoding human presenilin-2 protein is a mutant gene.

24. A method for identifying whether a compound is capable of ameliorating a neurodegenerative condition in an animal comprising:
(a) administering the compound to the transgenic animal of claim 10, wherein the animal exhibits a neurodegenerative condition;
(b) measuring the level of neurodegeneration in the animal; and
(c) comparing the level of neurodegeneration measured in step (b) with the level of neurodegeneration measured in the animal in the absence of the compound so as to identify whether the compound is capable of ameliorating the neurodegenerative condition in the animal.

25. The method of claim 24, wherein the neurodegenerative condition is associated with Alzheimer's disease, diabetes, senility, renal failure, hyperlipidemic atherosclerosis, neuronal cytoxicity, Down's syndrome, dementia associated with head trauma, amyotrophic lateral sclerosis, myasthenia gravis, multiple sclerosis or neuronal degeneration.

26. The method of claim 24, wherein the neurodegenerative condition is associated with degeneration of a neuronal cell in the subject.

27. The method of claim 24, wherein the neurodegenerative condition is associated with the formation of an amyloid-β peptide fibril.

28. The method of claim 24, wherein the neurodegenerative condition is associated with aggregation of amyloid-β peptide.

29. The method of claim 24, wherein the neurodegenerative condition is associated with infiltration of a microglial cell into a senile plaque.

30. The method of claim 24, wherein the neurodegenerative condition is associated with activation of a microglial cell by an amyloid-β peptide.

31. A cell comprising a recombinant nucleic acid which comprises DNA encoding mutant presenilin-2 protein and encoding receptor for advanced glycation end product protein.

32. The cell of claim 31 wherein the cell secretes mutant presenilin-2 and RAGE is transmembrane.

33. The cell of claim 31 wherein the cell is a neuronal cell, an endothelial cell, a glial cell, a microglial cell, an astrocyte, a smooth muscle cell, a somatic cell, a bone marrow cell, a liver cell, an intestinal cell, a germ cell, a myocyte, a mononuclear phagocyte, an endothelial cell, a tumor cell, a stem cell, or a PC12 cell.
